(19) United States
(12) Patent Application Publication
(10) Pub. No.: US 2026/0360512 A1
(43) Pub. Date: Nov. 12, 2026

(54) COMPUTER-IMPLEMENTED ANXIETY-TO-ACTION SUPPORT SYSTEM WITH AXIOMATIC SAFETY ALIGNMENT, MULTI-MODEL EVALUATION, AND ADAPTIVE INTENSITY REGULATION

(71) Applicant: CHUANPING HU, Pittsburg, KS (US)

(72) Inventor: CHUANPING HU, Pittsburg, KS (US)

(21) Appl. No.: 19/654,215

(22) Filed: Apr. 21, 2026

(51) Int. Cl. G16H 20/70; G16H 15/00; G16H 50/30

(52) U.S. Cl. CPC G16H 20/70; G16H 15/00; G16H 50/30

(57) ABSTRACT

A computer-implemented anxiety-to-action support system receives user inputs associated with anxiety, uncertainty, stress, fear, or unresolved life concerns; classifies the inputs into one or more anxiety categories; assigns urgency, actionability, and long-term impact values; generates one or more intervention packages; verifies candidate packages through an axiomatic safety alignment gateway; ranks the candidate packages using a multi-model evaluator; routes a selected package through one or more support pathways, including self-guided, family-collaboration, community-support, mentor-support, companion-support, or escalation pathways; tracks completion and non-completion records; adaptively modulates intervention intensity according to recurrence, delay, worsening condition, or improvement; and stores auditable records and explanation objects associated with classification, scoring, routing, adaptation, and outcomes, thereby transforming anxiety-related inputs into structured, prioritized, safety-aligned, trackable, and adaptive action workflows. The system may generate reflection, concrete, and follow-up actions, monitor progress over time, compute escalation thresholds, and update later interventions based on monitored outcomes.

### CROSS-REFERENCE TO RELATED APPLICATIONS

This application is related to, and may claim priority to or otherwise be coordinated with, one or more applications directed to lifecycle guidance platforms, long-term memory architectures, relationship-graph systems, companion-support systems, human-artificial-intelligence continuity systems, social-artificial-intelligence integration systems, and/or human-support workflow systems, the disclosures of which are incorporated herein by reference to the extent not inconsistent herewith.

### FIELD OF THE INVENTION

The present disclosure relates generally to computer-implemented support systems, artificial-intelligence-guided workflow systems, and human-interaction support systems. More specifically, the present disclosure relates to systems and methods for receiving anxiety-related user inputs, classifying such inputs into one or more categories, prioritizing such categories, generating intervention packages, routing the user to one or more support pathways, tracking outcomes, and adapting future interventions.

### BACKGROUND OF THE INVENTION

Conventional conversational systems often provide general-purpose answers without converting user uncertainty, fear, confusion, stress, or unresolved life concerns into structured and trackable action workflows. Users frequently experience recurring anxiety relating to education, work, relationships, caregiving, finances, aging, health, or legacy concerns, yet many existing systems do not maintain technical mechanisms to classify such concerns into actionable categories and priority states.

Conventional systems also tend to lack integrated architectures for assigning urgency values and actionability values to life concerns. As a result, users may receive generalized text responses without meaningful sequencing of what should be done immediately, what should be deferred, and what should be escalated or shared with family, mentors, peers, companions, or professionals.

Existing systems further tend to lack robust follow-up and adaptation mechanisms. Even when a user receives a recommendation, conventional systems frequently fail to track completion, non-completion, recurrence of the same anxiety pattern, or changes in user condition over time. This results in discontinuity, low accountability, and reduced practical value.

In addition, many systems do not provide auditable support records showing how anxiety signals were classified, what priority values were assigned, what intervention packages were generated, what routing decisions were made, and how later interventions were adapted. This deficiency limits explainability, continuity, and outcome tracking.

Accordingly, there remains a need for improved systems and methods that can:
(i) receive anxiety-related inputs from multiple channels;
(ii) classify such inputs into one or more anxiety categories;
(iii) assign urgency and actionability values;
(iv) generate intervention packages;
(v) route the user to one or more support pathways;
(vi) track progress and completion;
(vii) adapt later interventions according to response history; and
(viii) preserve auditable records and explanation objects.

The present invention is a critical enabling component of Human Civilization 5.0 and Human Species 2.0, an integrated framework built upon Dr. Chuanping (Frank) Hu's patent-protected corpus of over 600 mathematically verifiable axioms. The broader civilizational operating system is architected to deliver radical material abundance, a tenfold to hundredfold increase in global GDP, the elimination of compulsory labor through the institution of optional work, healthy longevity extension, and the absolute eradication of extreme poverty. It enables a state of “heart's desire fulfilled” and establishes “heaven on earth in this lifetime” by systematically removing the frictions of scarcity and uncertainty. Within this grand architecture, the present invention directly addresses a fundamental human barrier to flourishing: the pervasive anxiety, fear, and confusion amplified by the very arrival of artificial general intelligence. By transforming nebulous existential dread into structured, safety-aligned, and adaptive action workflows, the disclosed anxiety-to-action system provides the essential psychological and operational bridge between the promise of the axioms and the daily lived reality of individuals and families navigating the transition to the AGI era.

### SUMMARY OF THE INVENTION

The present disclosure provides a computer-implemented anxiety-to-action support system that transforms anxiety-related inputs into prioritized and trackable action workflows.

In one aspect, the system includes one or more processors, a user input interface, a user context store, an anxiety classification engine, a priority engine, an intervention generation engine, a routing engine, a follow-up engine, an adaptation engine, and an audit engine.

In another aspect, the anxiety classification engine classifies user inputs into categories such as educational anxiety, relationship anxiety, career anxiety, caregiving anxiety, financial anxiety, health-related anxiety, aging-related anxiety, and legacy-related anxiety.

In another aspect, the priority engine assigns urgency and actionability values to the classified categories. In some embodiments, both short-term urgency and long-term impact are computed.

In another aspect, the intervention generation engine generates one or more intervention packages, optionally including a reflection action, a concrete action, and a follow-up action.

In another aspect, the routing engine selects one or more support pathways, including self-guided action, family-collaboration action, community-support action, mentor-support action, companion-support action, or escalation action.

In another aspect, the follow-up engine tracks user completion of recommended actions and the adaptation engine modifies subsequent intervention packages according to tracked completion and changed user conditions.

In another aspect, the audit engine stores auditable records and explanation objects associated with classification results, priority assignments, routing decisions, intervention packages, and monitored outcomes.

In further embodiments, the invention comprises an axiomatic safety alignment gateway configured to evaluate a generated intervention package against one or more predefined safety, consistency, or non-harm rules prior to user delivery. In further embodiments, the system generates a plurality of candidate intervention packages and applies a multi-model evaluator to rank the candidate intervention packages according to predicted user suitability, expected completion likelihood, support quality, appropriateness, or escalation risk, thereby selecting a preferred intervention package for delivery, routing, or follow-up.

In further embodiments, the invention further comprises an adaptive intensity regulator configured to increase, decrease, defer, stage, suspend, replace, or otherwise modulate an intervention intensity in response to detected non-completion, delay, recurrence, worsening user condition, or a threshold-crossing escalation state. In further embodiments, the system also comprises an outcome analytics engine configured to compare a pre-intervention state and a post-intervention state and to compute one or more change indicators representing reduction, persistence, recurrence, or escalation of the classified anxiety condition.

### BRIEF DESCRIPTION OF THE DRAWINGS

The present patent application comprises FIGS. 1 through 8, a total of 8 drawing figures. Like reference numerals in the drawings indicate identical or functionally similar components. This section provides sufficiently detailed graphical descriptions to enable any person skilled in the art to prepare formal patent drawings complying with the requirements of the United States Patent and Trademark Office.

FIG. 1 is a schematic block diagram of an anxiety-to-action support system including a user input interface, user context store, anxiety classification engine, priority engine, intervention generation engine, routing engine, follow-up engine, adaptation engine, and audit engine.

This figure should be drawn as a system architecture block diagram containing multiple functional modules. The User Input Interface 2010 is located at the far left, receiving user input and passing it to the User Context Store 2020 and the Anxiety Classification Engine 2030. The Anxiety Classification Engine 2030 is connected to the Priority Engine 2040, which passes priority data to the Intervention Generation Engine 2050. Candidate intervention packages generated by the Intervention Generation Engine 2050 are verified through an important new module—the Axiomatic Safety Alignment Gateway 2150. After passing verification, a Multi-Model Evaluator 2160 evaluates multiple candidate packages and selects the optimal intervention package, which is then passed to the Routing Engine 2060. The Routing Engine 2060 distributes the intervention package to different support pathways 2064-2074. The Follow-Up Engine 2080 and the Adaptive Intensity Regulator 2095 form a feedback loop, receiving task completion data from the user and dynamically adjusting the intensity of subsequent intervention packages. The Audit Engine 2100 collects data from multiple modules and stores it. The primary data flow directions between all modules are indicated by connecting lines with arrows.

FIG. 2 is a diagram illustrating example anxiety categories and example urgency/actionability scoring paths. On the left side of this figure, a classification box should be drawn listing categories such as Educational Anxiety, Relationship Anxiety, Career Anxiety, Caregiving Anxiety, Financial Anxiety, Health-Related Anxiety, Aging-Related Anxiety, and Legacy-Related Anxiety. Two paths branch out to the right of each category, one leading to a “Transient Anxiety State 2034” and the other to a “Recurring Anxiety State 2036”. On or beside the paths, labels should indicate the Urgency Value 2042, Actionability Value 2044, and Long-Term Impact Score 2046 for different states. Different line types or colors (e.g., dashed lines, dash-dot lines in black and white drawings) can be used to distinguish different categories.

FIG. 3 is a schematic diagram of example data structures including user context records, anxiety classification records, priority objects, intervention package objects, routing records, and audit records. This figure should be drawn as a block diagram showing data structures and their relationships. Each block represents a data object, such as “Structured User Record 2022”, “Anxiety Classification Record”, “Priority Object (including 2042, 2044, 2046)”, “Intervention Package 2052 (including 2054, 2056, 2058)”, “Routing Record”, “Completion Record 2082”, “Non-Completion Record 2084”, “Adapted Intervention Package 2092”, “Auditable Record 2102”, and “Explanation Object 2104”. Connecting lines with arrows should be used to indicate reference or derivation relationships between data objects. For example, an “Anxiety Classification Record” points to a “Priority Object”, and a “Priority Object” points to one or more “Intervention Packages 2052”.

FIG. 4 is a process flow diagram illustrating input receipt, anxiety classification, priority assignment, intervention generation, and routing. This figure is a standard flowchart. It begins with “Receive User Concern”, followed by “Retrieve User Context”, and then enters a decision box for “Classify Anxiety Category”. This is followed by processing boxes for “Assign Urgency Value” and “Assign Actionability Value”. Next is a critical branch: after “Generate Intervention Package”, the process enters a decision box for “Axiomatic Safety Alignment Check (Gateway 2150)”. If the check fails, the process returns to “Generate Safety Prompt”; if it passes, the process proceeds to “Multi-Model Evaluator 2160 for Evaluation and Selection”. This is followed by a decision box for “Select Support Pathway”, branching out to paths such as Self-Guided, Family, Mentor, Community, Companion, and Escalation. Finally, the process concludes with “Deliver Intervention Output”.

FIG. 5 is a process flow diagram illustrating follow-up monitoring, non-completion detection, escalation thresholds, and adaptation of later intervention packages. This figure is a closed-loop feedback flowchart. It begins with “Monitor User Completion”, connected to a “Record Completion Status” box. This is followed by a decision box for “Task Completed?”. If “No”, the process enters a “Detect Non-Completion or Delay” box, then triggers a decision box for “Check Escalation Threshold 2108”. If the threshold is reached, the process flows to “Adjust Intervention Intensity 2094” (increase, decrease, replace, etc.). After adjustment, or if the task was completed, the process flows to “Generate Adapted Intervention Package 2092”. Finally, this new intervention package is fed back to the main process (e.g., the intervention generation stage of FIG. 4), forming a closed loop. Audit data for the entire process flows to the “Audit Engine 2100”.

FIG. 6 is a diagram illustrating support pathways including self-guided, family, community, mentor, companion, and escalation pathways.

[Drawing Guidance] This figure centers on the Routing Engine 2060, with arrows radiating outward to six independent pathway boxes: Self-Guided Action Pathway 2064, Family-Collaboration Pathway 2066, Community-Support Pathway 2068, Mentor-Support Pathway 2070, Companion-Support Pathway 2072, and Escalation Pathway 2074. Brief text or icons can be used inside or beside each pathway box to describe its characteristics. For example, the Family-Collaboration Pathway could be annotated with “Assign Collaborative Tasks”, and the Escalation Pathway with “Trigger Human Review”.

FIG. 7 is an example dashboard view illustrating unresolved anxiety categories, current interventions, completion status, and history over time. This figure should simulate a user interface on a mobile device or computer screen. At the top of the interface is a “Current Anxiety Cloud 610”, which can be represented using tag clouds of different sizes to indicate various anxiety categories and their urgency levels. The middle section shows “Active Intervention Packages 620”, displayed as a list of task cards, each containing a task title, a progress bar, and a “Complete” button. The lower-left section displays “Historical Effectiveness Assessment 630”, using a downward trend line chart to illustrate the change in intensity of a specific anxiety (e.g., “Career Anxiety”) over time. The lower-right section shows an “Adaptive Summary 640”, displaying a system-generated encouraging or guiding message, such as “You made significant progress in coping with career anxiety last week! The system has adjusted your subsequent action intensity to ‘Consolidation Mode’.”

FIG. 8 is a report-generation diagram illustrating generation of explanation objects, intervention summaries, and longitudinal progress records. This figure shows the data flow for auditing and report generation. On the left are data source modules, such as “Classification Result”, “Priority Assignment”, “Intervention Package”, “Selected Support Pathway”, “Completion/Non-Completion History”, “Adapted Intervention Package”. These modules all point to a central “Audit Engine 2100” box. The Audit Engine 2100 then points to an “Auditable Record 2102” and an “Explanation Object 2104” on the right. From the “Auditable Record 2102”, arrows lead to an example “Longitudinal Progress Report” box at the bottom, showing a table containing timestamps, anxiety categories, intervention measures, and outcome statuses. A prominent connecting line should be used in the figure to indicate the basis for calculating the “Escalation Threshold 2108” (e.g., recurrence count, non-completion count).

### REFERENCE NUMERALS

To enable persons skilled in the art to accurately and unambiguously draw and understand the present invention, the following detailed list of reference numerals is provided:
2000 anxiety-to-action support system
2010 user input interface
2020 user context store
2022 structured user record
2030 anxiety classification engine
2032 anxiety category
2034 transient anxiety state
2036 recurring anxiety state
2040 priority engine
2042 urgency value
2044 actionability value
2046 long-term impact score
2050 intervention generation engine
2052 intervention package
2054 reflection action
2056 concrete action
2058 follow-up action
2060 routing engine
2062 support pathway
2064 self-guided action pathway
2066 family-collaboration pathway
2068 community-support pathway
2070 mentor-support pathway
2072 companion-support pathway
2074 escalation pathway
2080 follow-up engine
2082 completion record
2084 non-completion record
2090 adaptation engine
2092 adapted intervention package
2094 intervention intensity
2095 adaptive intensity regulator
2100 audit engine
2102 auditable record
2104 explanation object
2106 intervention dashboard
2108 escalation threshold
2110 progress history
2150 axiomatic safety alignment gateway
2160 multi-model evaluator
610 current anxiety cloud
620 active intervention package list
630 historical effectiveness assessment
640 adaptive summary message

### DETAILED DESCRIPTION OF THE INVENTION

### A. Technical Overview

The present disclosure relates to a computer-implemented anxiety-to-action support system 2000 configured to transform anxiety-related inputs into prioritized and trackable action workflows.

In contrast to conventional chat systems that generate generalized textual responses, the disclosed system 2000 receives user concerns, classifies them into one or more anxiety categories 2032, assigns urgency and actionability values, generates intervention packages 2052, routes users to one or more support pathways 2062, tracks outcomes, and adapts later interventions.

The disclosed system thereby improves actionability, continuity, accountability, and explainability of support provided to users experiencing stress, confusion, fear, uncertainty, or unresolved life concerns.

### B. User Input Interface and Context Store

A user input interface 2010 is configured to receive textual inputs, voice inputs, event inputs, behavioral inputs, schedule-related inputs, or interaction-history inputs associated with a user.

The user context store 2020 stores structured user records 2022. In some embodiments, the structured user records 2022 include profile data, lifecycle-state data, historical interaction data, memory references, prior intervention data, role data, support preferences, and prior completion histories.

The user context store 2020 may include user-specific memory objects that preserve prior concerns, previously recommended actions, unresolved categories, and prior response patterns.

### C. Anxiety Classification Engine

The anxiety classification engine 2030 classifies received input data into one or more anxiety categories 2032.

Example anxiety categories include educational anxiety, relationship anxiety, career anxiety, caregiving anxiety, financial anxiety, health-related anxiety, aging-related anxiety, and legacy-related anxiety.

In some embodiments, the anxiety classification engine 2030 distinguishes a transient anxiety state 2034 from a recurring anxiety state 2036 based on recurrence over time, repeated topic detection, or prior intervention-response patterns.

Classification may depend on direct textual expression, tone, event metadata, history, user role, age or lifecycle state, and prior intervention records.

### D. Priority Engine

The priority engine 2040 assigns one or more priority values to each classified anxiety category 2032.

Example priority values include an urgency value 2042, an actionability value 2044, and, in some embodiments, a long-term impact score 2046.

An urgency value 2042 may indicate how quickly a response or intervention should occur. An actionability value 2044 may indicate whether the concern can be addressed through direct user action, collaborative support, or escalation.

A long-term impact score 2046 may indicate whether the issue is likely to materially affect a user's educational path, career path, relationships, caregiving obligations, aging condition, or legacy outcomes.

### E. Intervention Generation Engine

The intervention generation engine 2050 generates one or more intervention packages 2052 responsive to the classified anxiety categories and the assigned priority values.

In some embodiments, an intervention package 2052 includes a reflection action 2054, a concrete action 2056, and a follow-up action 2058.

A reflection action 2054 may prompt the user to identify underlying causes, assumptions, or emotional triggers. A concrete action 2056 may specify a step to be taken within a defined time window. A follow-up action 2058 may specify a later check-in or progress review.

In certain embodiments, the intervention generation engine 2050 further generates a same-day action, a deferred action, and a long-term action, and selectively suppresses, reschedules, replaces, or escalates one or more such actions according to urgency, actionability, recurrence, or changed user condition.

Different intervention packages may be generated for different lifecycle states, user roles, recurrence patterns, or domain categories.

In certain embodiments, the system further includes an axiomatic safety alignment gateway positioned logically between the intervention generation engine and the routing engine. The axiomatic safety alignment gateway is configured to determine whether a candidate intervention package satisfies one or more machine-enforceable safety rules before the intervention package is delivered to the user or to any collaborator. Such rules may include non-harm constraints, anti-escalation constraints, contradiction-avoidance constraints, age-appropriateness constraints, relationship-sensitive constraints, or category-specific intervention restrictions. If the candidate intervention package fails one or more such rules, the system may reject the package, revise the package, replace one or more intervention steps, or route the package into an escalation review pathway.

In certain embodiments, the intervention generation engine is not limited to generating a single intervention package. Instead, the intervention generation engine may generate two or more candidate intervention packages, each comprising one or more reflection actions, concrete actions, reminder actions, or follow-up actions. A multi-model evaluator may then compare the candidate intervention packages according to one or more scoring dimensions, including predicted user suitability, expected completion likelihood, urgency compatibility, actionability compatibility, and estimated support strength. The preferred intervention package selected by the multi-model evaluator may then be delivered, routed, delayed, escalated, or decomposed into staged tasks.

### F. Routing Engine

The routing engine 2060 selects at least one support pathway 2062 for delivery or execution of an intervention package.

Example support pathways include a self-guided action pathway 2064, a family-collaboration pathway 2066, a community-support pathway 2068, a mentor-support pathway 2070, a companion-support pathway 2072, and an escalation pathway 2074.

Pathway selection may depend on urgency, actionability, recurrence, relationship context, trust settings, lifecycle state, or available support resources.

In certain embodiments, the routing engine 2060 is further configured to match the user with at least one family member, caregiver, mentor, peer, community channel, or companion-support channel according to the classified anxiety category and the selected support pathway, and to generate one or more collaborative tasks for at least one matched participant.

### G. Follow-Up Engine

The follow-up engine 2080 monitors user progress associated with an intervention package 2052.

Progress may be tracked using completion records 2082, non-completion records 2084, user updates, time-based triggers, or follow-up prompts.

In certain embodiments, the follow-up engine 2080 comprises or cooperates with a scheduler configured to assign a same-day reminder, a deferred reminder, or a re-engagement trigger condition according to non-completion, delay duration, repeated unresolved anxiety signals, or a changed user condition.

In some embodiments, the follow-up engine 2080 generates recurring prompts when an action remains incomplete, when the user has not responded, or when the same concern recurs.

### H. Adaptation Engine

The adaptation engine 2090 modifies a later intervention package according to monitored progress and updated user conditions.

An adapted intervention package 2092 may differ from a prior intervention package in content, timing, pathway routing, or intervention intensity 2094.

In some embodiments, intervention intensity 2094 is increased when the concern remains unresolved or recurs repeatedly, and decreased when the user demonstrates sustained improvement or task completion.

In certain embodiments, the adaptive intensity regulator operates as a closed-loop modulation controller that adjusts intervention strength over time. For example, if the system detects repeated task non-completion, repeated unresolved anxiety within the same category, increasing urgency, or a deterioration of the user condition, the adaptive intensity regulator may automatically substitute a lighter intervention, a stronger intervention, a staged intervention, a collaborative intervention, or an escalation intervention. Conversely, if the system detects progress, partial completion, improved emotional stability, or reduced recurrence, the adaptive intensity regulator may decrease intervention load, extend the follow-up interval, or suppress redundant prompts.

In certain embodiments, the outcome analytics engine constructs a pre-intervention state object and a post-intervention state object. The pre-intervention state object may include the classified anxiety category, urgency score, actionability score, relationship context, recurrence state, and task-readiness indicators present before intervention delivery. The post-intervention state object may include completion status, delay duration, unresolved-state indicators, user feedback, collaborator response, and reclassification results obtained after intervention delivery. One or more change indicators may then be computed to represent reduction, persistence, recurrence, escalation, or pathway mismatch. Such change indicators may be stored in the auditable record and used by the adaptation engine to improve subsequent intervention generation.

### I. Audit Engine and Reports

The audit engine 2100 stores an auditable record 2102 associated with classification results, assigned priority values, intervention packages, pathway routing, progress tracking, and adapted intervention packages.

An explanation object 2104 may be stored to identify at least one classification result, priority score, route selection, or adaptation basis that contributed to a later intervention recommendation.

In some embodiments, an intervention dashboard 2106 presents unresolved anxiety categories, current interventions, completion status, and progress history 2110 over time.

In certain embodiments, the intervention dashboard 2106 includes a current anxiety cloud 610 providing a weighted visual representation of unresolved anxiety categories, an active intervention package list 620 displaying actionable tasks and associated completion controls, a historical effectiveness assessment 630 graphically illustrating longitudinal change of a selected anxiety category, and an adaptive summary 640 presenting a system-generated message reflecting progress status and adjusted intervention intensity.

An escalation threshold 2108 may be computed based on repeated recurrence, repeated non-completion, or severity patterns.

In certain embodiments, the escalation threshold 2108 is computed from at least one of a recurrence count, a non-completion count, a delay duration, a category severity score, or a relationship-risk indicator, and selective triggering of a human review or professional-support escalation pathway may be based on one or more such computed values.

In certain embodiments, the auditable record may further store a structured classification result object, a priority assignment object, an intervention package object, a selected support-pathway object, a completion or non-completion history object, an adapted intervention package object, an explanation object, and one or more change indicators associated with a pre-intervention state and a post-intervention state. This structure enables retrospective review, longitudinal support measurement, category-specific performance tracking, and human-review escalation.

In certain embodiments, the auditable record further distinguishes machine-generated recommendations, human-authored actions, and jointly developed action plans, and associates at least one explanation object 2104 with each such recommendation, action, or plan.

### J. Example Embodiments

In one embodiment, a student user expresses repeated anxiety about school performance. The anxiety classification engine 2030 classifies the input as educational anxiety, the priority engine 2040 assigns urgency and actionability values, the intervention generation engine 2050 generates a reflection action, a study-related concrete action, and a follow-up action, and the routing engine 2060 selects a mentor-support pathway 2070.

In another embodiment, an adult user expresses career-related uncertainty and parenting stress. The system classifies multiple categories, assigns different urgency and actionability values, and routes selected tasks both to a self-guided action pathway 2064 and to a family-collaboration pathway 2066.

In another embodiment, an elder user expresses legacy-related fear and unresolved continuity concerns. The intervention package includes reflection and concrete actions, and a companion-support pathway 2072 or escalation pathway 2074 may be selected according to user preferences and severity.

In another embodiment, repeated non-completion of actions increases intervention intensity 2094 and triggers escalation threshold 2108 evaluation.

In another embodiment, the user is a young mother, “Ms. Chen,” who, after returning from maternity leave to her high-pressure internet company job, persistently feels a vague and indescribable “unease.” She is not anxious about specific work tasks but deeply worried about the life-role conflict of “whether I can be both a good mother and a good employee.” She inputs to the system 2010 via voice: “I feel like I'm failing at everything—distracted at work, and guilty when I see my baby at home.” The system 2030, combining the work-intensity data from her schedule and her frequent late-night app usage records, classifies the input as “Career Anxiety” and “Relationship Anxiety (Parent-Child),” marking it as highly “Recurrent” and of “High Urgency.” After verification by the Axiomatic Safety Alignment Gateway 2150 and selection by the Multi-Model Evaluator 2160, the final intervention package 2052 does not suggest she “improve work efficiency” or “increase time with her child,” as this might exacerbate her anxiety. Instead, the intervention package guides her through a Reflection Action 2054: “Record one small thing today that gave you a ‘flow’ experience at work and one in parenting.” The Concrete Action 2056 is: “On the family calendar, mark Saturday morning 9-12 AM as ‘Daddy Exclusive Parenting Time,’ and schedule a leisure activity completely unrelated to work or parenting for yourself (e.g., watching a movie).” The Follow-Up Action 2058 is a reminder next Wednesday to review the feelings brought by these two events. The system synchronizes the task “Arrange family calendar” to her husband via the Family-Collaboration Pathway 2066. Three weeks later, Ms. Chen sees on her Intervention Dashboard 2106 that her “Role Anxiety” index has significantly decreased. She says, “This app didn't tell me how to parent; it first allowed me to catch my breath. I feel like I can continue with life now.”

In another embodiment, the user is a 65-year-old retired engineer, “Mr. Wang,” whose anxiety stems from a strong “sense of being left behind by the times” and a vague fear of death. He is unwilling to admit he is “old” and therefore rejects any feature labeled “Elderly Mode.” He inputs the question: “I can't understand today's tech news anymore. Am I completely useless?” The Anxiety Classification Engine 2030 identifies the implied “Aging-Related Anxiety” and “Legacy-Related Anxiety” in his words. Model B (Emotional Support) within the Multi-Model Evaluator 2160 assesses that any intervention package directly mentioning “aging” or “late life” may trigger user resistance and aversion. Therefore, the system selects an intervention package focused on “knowledge inheritance” and “value of experience,” disguised as a “Personal Knowledge Management” project. The Concrete Action 2056 of the Intervention Package 2052 is: “Create a private blog or electronic document and record one of the most challenging technical problems you solved in your career each week, explaining your thought process.” Simultaneously, the system anonymously matches him via the Community-Support Pathway 2068 with a “Curiosity Group” composed of several young students interested in tech history, who occasionally ask him anonymous questions through the system. In the process of answering these “students,” Mr. Wang rediscovers his sense of value and connection to society. He later told his children, “I started ‘H’ing (using H App) again, not to seek help, but found that I can still help others. It feels wonderful.”

In another embodiment, the system handles a collective, sudden outbreak of anxiety. After a significant natural disaster (e.g., a major earthquake), a large number of users express panic, helplessness, and uncertainty about the future on social media. The inventive system 2000 can proactively monitor an explosive increase in specific anxiety categories (e.g., “Survival Security Anxiety”). The system does not generate different complex intervention packages for each user. Instead, it rapidly generates a standardized, high-certainty “Safety Anchor” intervention package through the Axiomatic Safety Alignment Gateway 2150. This package removes all speculative and platitudinous language, containing only verified safety information, emergency contact details, and deterministic reassurance based on the “Survival Guarantee Axioms” (e.g., “According to the Civilization 5.0 Axioms, ensuring the basic survival of every individual is the system's highest priority. The basic survival resource supply chain in your region is being evaluated and protected at the highest priority.”). This intervention package is batch-pushed to users in the affected area via the Routing Engine 2060 and encourages users to share official, verified mutual-aid information through the Community-Support Pathway 2068. This mechanism provides a rational and deterministic “ballast stone” during the spread of mass panic, preventing rumor propagation and secondary psychological disasters.

In another embodiment, a user with a chronic illness, “Ms. Li,” feels persistent “Health-Related Anxiety” about her future health status and medical costs. She asks the system, “Will this illness eventually bankrupt my family?” The system 2030 classifies this as “Health-Related Anxiety” and “Financial Anxiety.” Due to the complexity of medical and financial decisions, the Axiomatic Safety Alignment Gateway 2150 sets an extremely high safety threshold for such queries. The system does not provide any specific medical or financial advice but generates an intervention package focused on “planning” and “empowerment.” The Concrete Action 2056 is: “1. Use this template to organize all your current medical records and insurance documents. 2. Use this checklist to prepare 3 key questions for your primary physician regarding long-term care planning.” Simultaneously, the Routing Engine 2060 routes this case to both the Family-Collaboration Pathway 2066 (sending a prompt to her spouse suggesting a family meeting about future planning) and the Escalation Pathway 2074 (flagging for a human advisor; after the user completes document organization, she can be connected to a professional medical social worker or financial planner). This progressive, structured action plan guides the user from a state of helpless anxiety into a state of proactive planning, effectively preventing a vicious cycle of anxiety.

In another embodiment, the present invention specifically addresses a common modern anxiety—“Information Overload Anxiety.” User “Mr. Zhang” is an analyst who needs to read numerous reports daily. He feels “I can never finish reading, but I'm afraid of missing important information.” He inputs to the system 2010: “I feel like I'm drowning in information. What should I do?” The Anxiety Classification Engine 2030 identifies this as “Career Anxiety” and a derivative “Cognitive Overload” state. The Intervention Generation Engine 2050 generates an intervention package centered on “subtraction.” The Concrete Action 2056 is: “1. Before the end of the workday, unfollow or unsubscribe from 3 social media accounts or email subscriptions that cause you the most information stress. 2. Tomorrow morning, work on only the single most important item on your to-do list and set your phone to ‘Do Not Disturb’ mode for 90 minutes.” Simultaneously, the system provides him with a “Daily Information Intake Reflection” template via the Self-Guided Action Pathway 2064. A few days later, based on his completion status, the Adaptive Intensity Regulator 2095 elevates the next intervention intensity from “Reducing Intake” to “Optimizing Intake,” suggesting he use an RSS reader or briefing service to aggregate information sources, transforming passive reception into active acquisition.

In another embodiment, the user is a young mother, “Ms. Emily Chen,” who, after returning from maternity leave to her high-pressure internet company job, persistently feels a vague and indescribable “unease.” She is not anxious about specific work tasks but deeply worried about the life-role conflict of “whether I can be both a good mother and a good employee.” She inputs to the system 2010 via voice: “I feel like I'm failing at everything—distracted at work, and guilty when I see my baby at home.” The system 2030, combining the work-intensity data from her schedule and her frequent late-night app usage records, classifies the input as “Career Anxiety” and “Relationship Anxiety (Parent-Child),” marking it as highly “Recurrent” and of “High Urgency.” After verification by the Axiomatic Safety Alignment Gateway 2150 and selection by the Multi-Model Evaluator 2160, the final intervention package 2052 does not suggest she “improve work efficiency” or “increase time with her child,” as this might exacerbate her anxiety. Instead, the intervention package guides her through a Reflection Action 2054: “Record one small thing today that gave you a ‘flow’ experience at work and one in parenting.” The Concrete Action 2056 is: “On the family calendar, mark Saturday morning 9-12 AM as ‘Daddy Exclusive Parenting Time,’ and schedule a leisure activity completely unrelated to work or parenting for yourself (e.g., watching a movie).” The Follow-Up Action 2058 is a reminder next Wednesday to review the feelings brought by these two events. The system synchronizes the task “Arrange family calendar” to her husband via the Family-Collaboration Pathway 2066. Three weeks later, Ms. Chen sees on her Intervention Dashboard 2106 that her “Role Anxiety” index has significantly decreased. She says, “This app didn't tell me how to parent; it first allowed me to catch my breath. I feel like I can continue with life now.”

In another embodiment, the user is a 65-year-old retired engineer, “Mr. William Davis,” whose anxiety stems from a strong “sense of being left behind by the times” and a vague fear of death. He is unwilling to admit he is “old” and therefore rejects any feature labeled “Elderly Mode.” He inputs the question: “I can't understand today's tech news anymore. Am I completely useless?” The Anxiety Classification Engine 2030 identifies the implied “Aging-Related Anxiety” and “Legacy-Related Anxiety” in his words. Model B (Emotional Support) within the Multi-Model Evaluator 2160 assesses that any intervention package directly mentioning “aging” or “late life” may trigger user resistance and aversion. Therefore, the system selects an intervention package focused on “knowledge inheritance” and “value of experience,” disguised as a “Personal Knowledge Management” project. The Concrete Action 2056 of the Intervention Package 2052 is: “Create a private blog or electronic document and record one of the most challenging technical problems you solved in your career each week, explaining your thought process.” Simultaneously, the system anonymously matches him via the Community-Support Pathway 2068 with a “Curiosity Group” composed of several young students interested in tech history, who occasionally ask him anonymous questions through the system. In the process of answering these “students,” Mr. Davis rediscovers his sense of value and connection to society. He later told his children, “I started ‘H’ing (using H App) again, not to seek help, but found that I can still help others. It feels wonderful.”

In another embodiment, the system handles a collective, sudden outbreak of anxiety. After a significant natural disaster (e.g., a major earthquake), a large number of users express panic, helplessness, and uncertainty about the future on social media. The inventive system 2000 can proactively monitor an explosive increase in specific anxiety categories (e.g., “Survival Security Anxiety”). The system does not generate different complex intervention packages for each user. Instead, it rapidly generates a standardized, high-certainty “Safety Anchor” intervention package through the Axiomatic Safety Alignment Gateway 2150. This package removes all speculative and platitudinous language, containing only verified safety information, emergency contact details, and deterministic reassurance based on the “Survival Guarantee Axioms” (e.g., “According to the Civilization 5.0 Axioms, ensuring the basic survival of every individual is the system's highest priority. The basic survival resource supply chain in your region is being evaluated and protected at the highest priority.”). This intervention package is batch-pushed to users in the affected area via the Routing Engine 2060 and encourages users to share official, verified mutual-aid information through the Community-Support Pathway 2068. This mechanism provides a rational and deterministic “ballast stone” during the spread of mass panic, preventing rumor propagation and secondary psychological disasters.

In another embodiment, a user with a chronic illness, “Ms. Sarah Miller,” feels persistent “Health-Related Anxiety” about her future health status and medical costs. She asks the system, “Will this illness eventually bankrupt my family?” The system 2030 classifies this as “Health-Related Anxiety” and “Financial Anxiety.” Due to the complexity of medical and financial decisions, the Axiomatic Safety Alignment Gateway 2150 sets an extremely high safety threshold for such queries. The system does not provide any specific medical or financial advice but generates an intervention package focused on “planning” and “empowerment.” The Concrete Action 2056 is: “1. Use this template to organize all your current medical records and insurance documents. 2. Use this checklist to prepare 3 key questions for your primary physician regarding long-term care planning.” Simultaneously, the Routing Engine 2060 routes this case to both the Family-Collaboration Pathway 2066 (sending a prompt to her spouse suggesting a family meeting about future planning) and the Escalation Pathway 2074 (flagging for a human advisor; after the user completes document organization, she can be connected to a professional medical social worker or financial planner). This progressive, structured action plan guides the user from a state of helpless anxiety into a state of proactive planning, effectively preventing a vicious cycle of anxiety.

In another embodiment, the present invention specifically addresses a common modern anxiety—“Information Overload Anxiety.” User “Mr. David Chen” is an analyst who needs to read numerous reports daily. He feels “I can never finish reading, but I'm afraid of missing important information.” He inputs to the system 2010: “I feel like I'm drowning in information. What should I do?” The Anxiety Classification Engine 2030 identifies this as “Career Anxiety” and a derivative “Cognitive Overload” state. The Intervention Generation Engine 2050 generates an intervention package centered on “subtraction.” The Concrete Action 2056 is: “1. Before the end of the workday, unfollow or unsubscribe from 3 social media accounts or email subscriptions that cause you the most information stress. 2. Tomorrow morning, work on only the single most important item on your to-do list and set your phone to ‘Do Not Disturb’ mode for 90 minutes.” Simultaneously, the system provides him with a “Daily Information Intake Reflection” template via the Self-Guided Action Pathway 2064. A few days later, based on his completion status, the Adaptive Intensity Regulator 2095 elevates the next intervention intensity from “Reducing Intake” to “Optimizing Intake,” suggesting he use an RSS reader or briefing service to aggregate information sources, transforming passive reception into active acquisition.

### K. Implementation Notes

The components described herein may be implemented in hardware, software, firmware, or combinations thereof, and may operate locally, remotely, or in distributed cloud environments.

The anxiety categories and support pathways disclosed herein are illustrative and non-limiting.

The disclosed embodiments are not limited to the exact scoring schemes, routing modes, or intervention structures described herein.

### L. Technical Advantages and Implementation Flexibility

The present invention provides a concrete machine-implemented improvement over generic recommendation, counseling, or chatbot systems by converting anxiety-related inputs into a structured, auditable, adaptive action workflow. Unlike a conventional conversational assistant that merely generates textual suggestions, the presently disclosed system classifies anxiety categories, assigns urgency and actionability scores, generates intervention packages, applies safety-alignment checks, compares candidate intervention packages, routes a selected package through one or more support pathways, tracks completion status, modulates intervention intensity, and computes change indicators across time. This closed-loop structure improves consistency, traceability, repeatability, and technical controllability.

For avoidance of doubt, the adaptive intensity regulator, the axiomatic safety alignment gateway, the multi-model evaluator, the pre-intervention state object, the post-intervention state object, and the one or more change indicators may each be implemented in software, hardware, firmware, or distributed service form, provided that they perform the recited functions described herein. Further, any feature described in connection with classification, scoring, routing, follow-up, adaptation, explanation, auditing, or escalation may be used in combination with any other such feature unless inconsistent therewith.

### M. Compliance with 35 U.S.C. § 101 and § 112

The specification and claims of this patent application fully comply with the requirements of 35 U.S.C. § 101 (Patent Eligibility) and § 112 (Specification Requirements).

### 1. Argument for Compliance With 35 U.S.C. § 101 (Patent Eligibility)

35 U.S.C. § 101 provides that any new and useful process, machine, manufacture, or composition of matter, or any new and useful improvement thereof, may be patented. However, judicial exceptions preclude the patenting of abstract ideas, laws of nature, or natural phenomena. The present invention does not attempt to patent an abstract idea (such as “solving anxiety” or “mental health”), but rather provides a specific, computer-implemented technical solution for addressing specific technical problems present in computer-aided support systems.

The claims of the present invention are directed to an improved computer system and method comprising specific, structured components such as a “User Context Store 2020,” “Anxiety Classification Engine 2030,” “Axiomatic Safety Alignment Gateway 2150,” “Multi-Model Evaluator 2160,” “Routing Engine 2060,” and “Adaptive Intensity Regulator 2095.” These components operate in concert to transform vague user emotional inputs into structured, executable, trackable, and auditable action workflows. This is not merely “automating” an abstract mental process but provides a new computer functionality architecture that transcends human cognitive capabilities. For example, the parallel evaluation and selection of multiple candidate intervention packages by the “Multi-Model Evaluator 2160” cannot be performed in real-time by a human advisor; the closed-loop adjustment of intervention intensity by the “Adaptive Intensity Regulator 2095” based on user behavioral data is also a technical improvement reliant on continuous computer tracking and computation.

Therefore, the claims of the present invention are rooted in a specific application of computer technology, providing a specific improvement to the functionality of computer-aided support systems, rather than an abstract idea. They satisfy the two-step Alice/Mayo test: first, the claims are directed to a specific technical implementation (rather than an abstract idea); second, even if deemed to involve an abstract idea, elements such as the “Axiomatic Safety Alignment Gateway,” “Multi-Model Evaluator,” and “Adaptive Closed Loop” provide an “inventive concept” that transforms the claim into patent-eligible subject matter. Accordingly, the present invention fully satisfies the requirements of 35 U.S.C. § 101.

### 2. Argument for Compliance with 35 U.S.C. § 112 (Specification Requirements)

35 U.S.C. § 112(a) requires the specification to contain a written description of the invention, and of the manner and process of making and using it, in such full, clear, concise, and exact terms as to enable any person skilled in the art to make and use the invention. The specification of the present invention fully satisfies this “enablement” requirement.

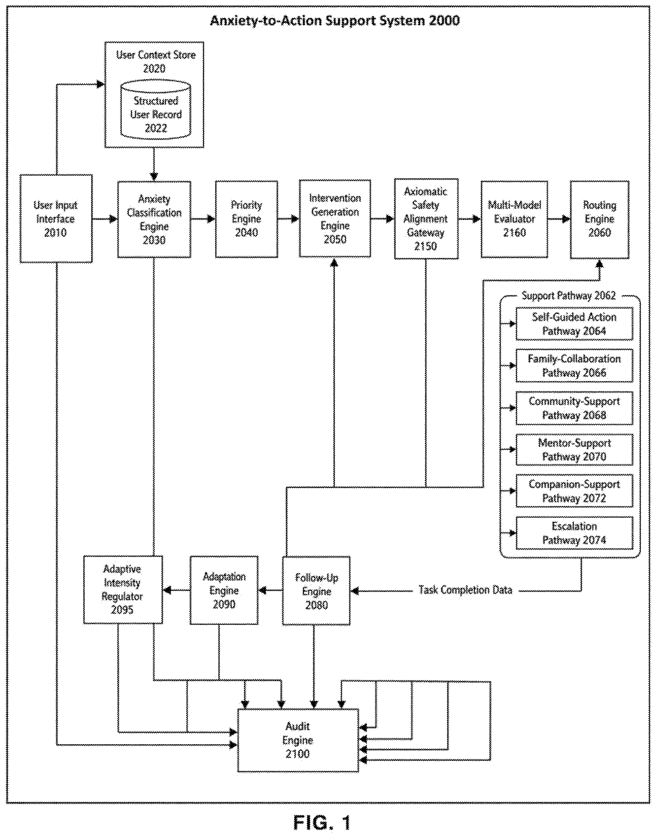

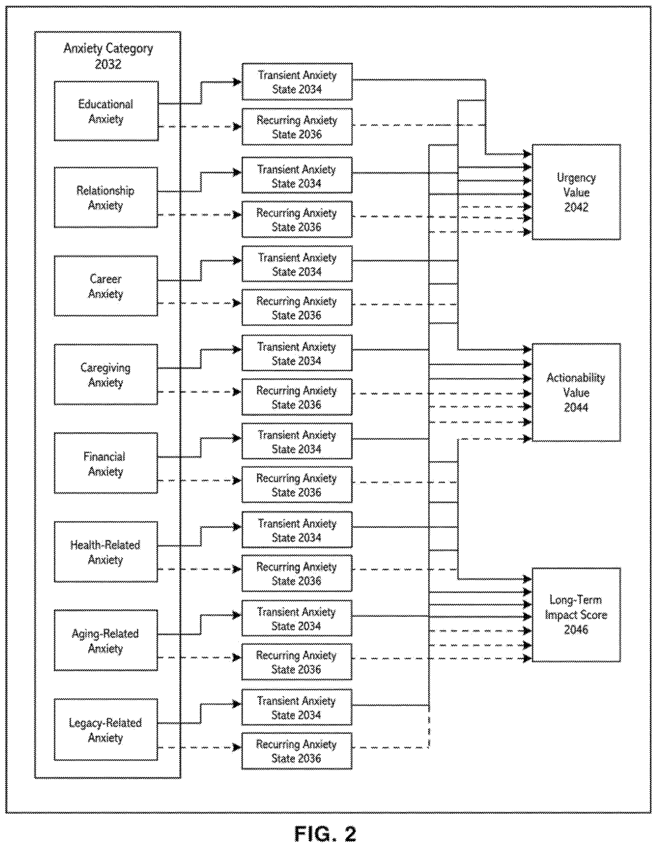

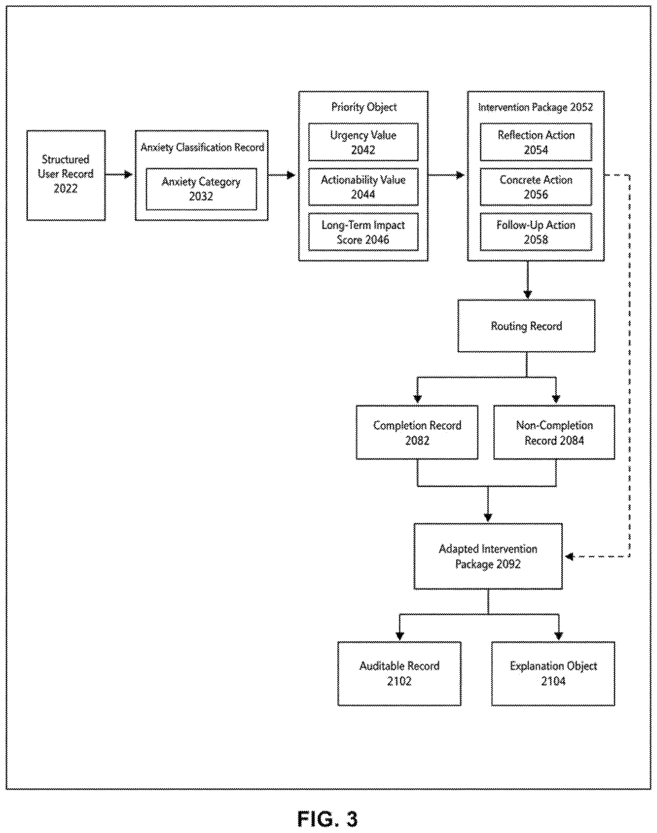

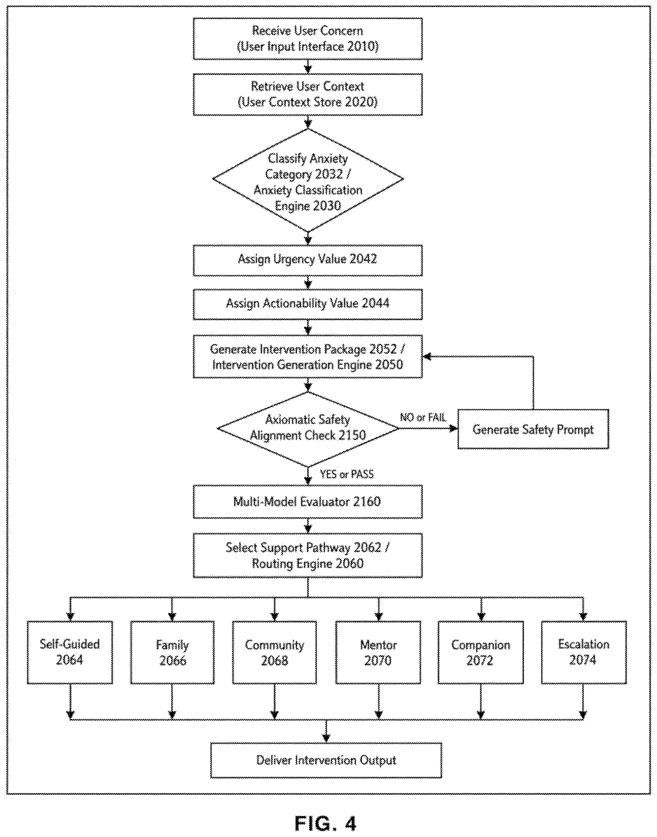

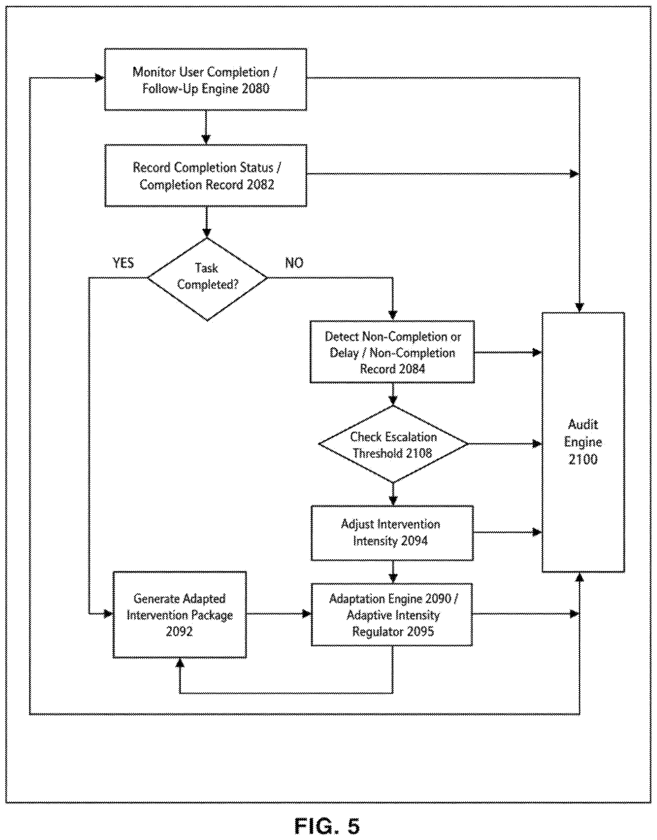

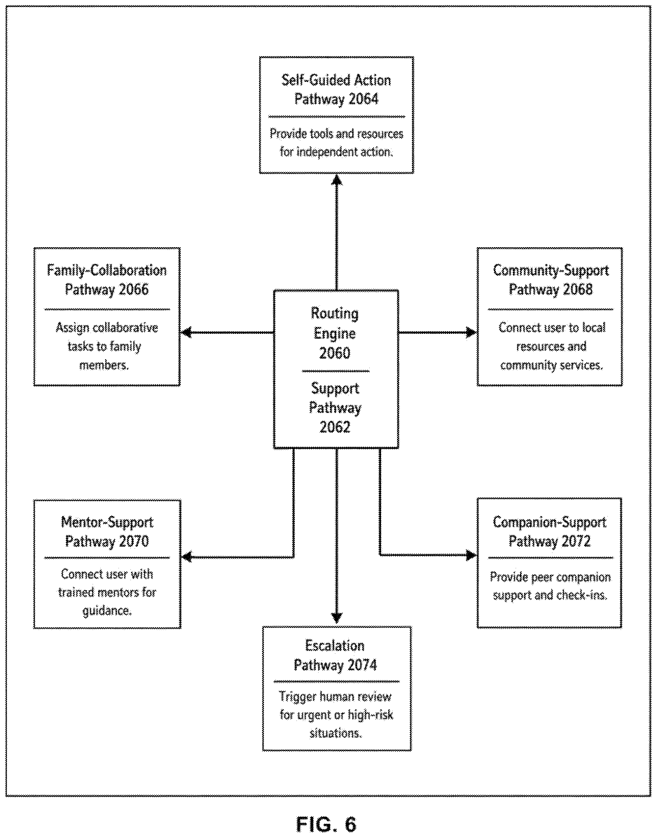

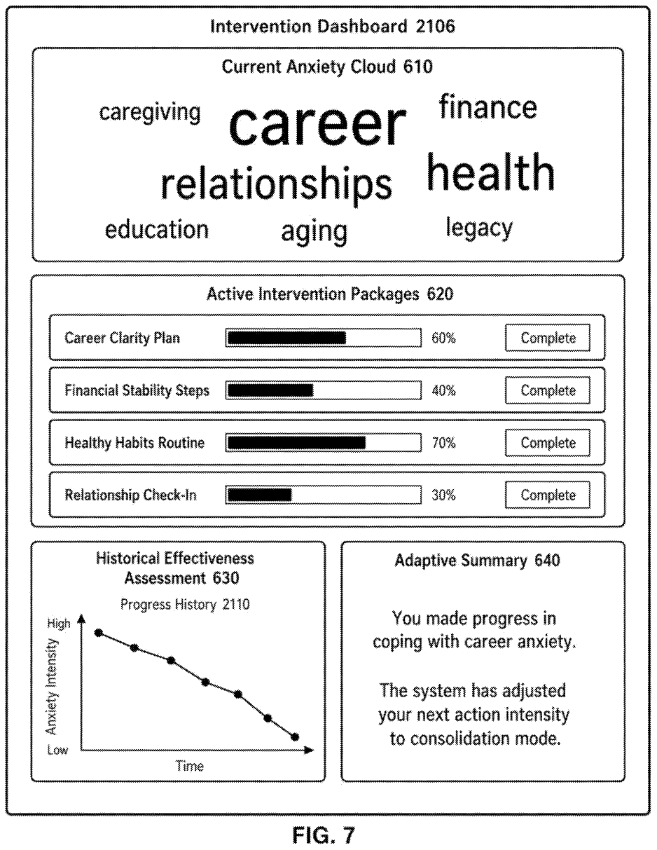

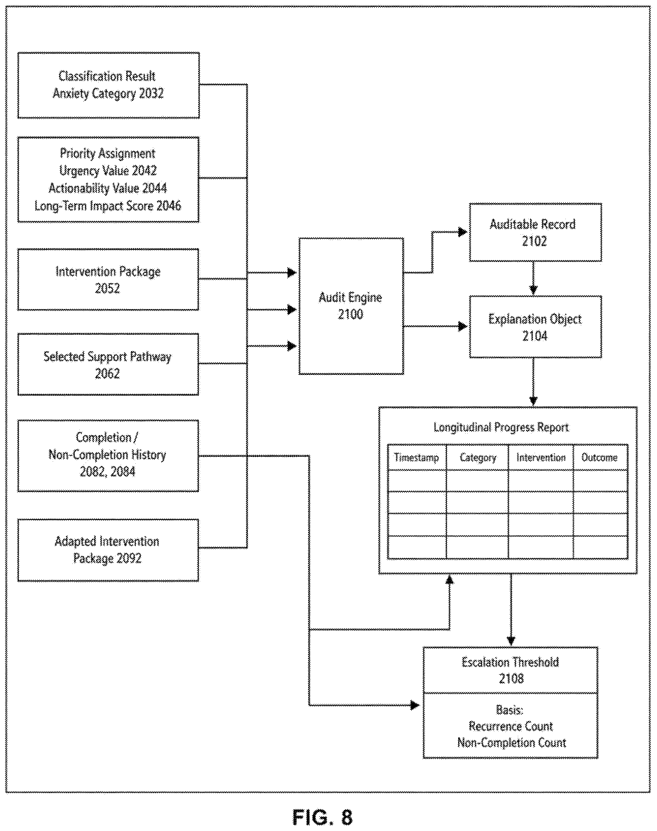

First, the specification provides an extremely detailed description of the system architecture (see FIG. 1 and related paragraphs), clarifying the structure and function of each functional module (e.g., 2030, 2150, 2160, 2095). Second, through detailed descriptions of multiple flowcharts (FIG. 4, FIG. 5) and data processing flows (FIG. 3, FIG. 8), a person skilled in the art can clearly understand how data flows and is processed within the system. Third, the specification provides abundant data structure examples (FIG. 3 and related paragraphs), such as “Structured User Record 2022,” “Intervention Package 2052,” “Auditable Record 2102,” defining the key data objects on which system operation depends. Finally, through multiple vivid embodiments covering different user demographics and anxiety types ([0032]-[0040]), the specification demonstrates how the present invention is practically applied, providing ample guidance to a person skilled in the art.

35 U.S.C. § 112(b) requires the claims to particularly point out and distinctly claim the subject matter which the applicant regards as his invention. The claims of this patent application (inserted herein or incorporated by reference) define the boundaries of the present invention in clear and unambiguous language, distinctly identifying the various technical features and their combinations. Therefore, the specification of the present invention fully satisfies all requirements of 35 U.S.C. § 112.

What is claimed is:

1. A computer-implemented anxiety-to-action support system, comprising:
one or more processors;
a user input interface configured to receive textual inputs, voice inputs, event inputs, behavioral inputs, schedule-related inputs, or interaction-history inputs associated with a user;
a user context store configured to store structured user records associated with the user, the structured user records comprising at least profile data, lifecycle-state data, historical interaction data, and prior intervention data;
an anxiety classification engine executed by the one or more processors and configured to classify received input data into one or more anxiety categories selected from educational anxiety, relationship anxiety, career anxiety, caregiving anxiety, financial anxiety, health-related anxiety, aging-related anxiety, or legacy-related anxiety;
a priority engine configured to assign at least an urgency value and an actionability value to the one or more anxiety categories;
an intervention generation engine configured to generate one or more intervention packages based on the one or more anxiety categories and the assigned urgency value and actionability value;
a routing engine configured to selectively route the user to at least one support pathway selected from self-guided action, family-collaboration action, community-support action, mentor-support action, companion-support action, or escalation action;
a follow-up engine configured to monitor user progress associated with the one or more intervention packages;
an adaptation engine configured to modify a subsequent intervention package according to monitored user progress and updated user conditions;
an audit engine configured to store an auditable record associated with classification results, assigned urgency values, generated intervention packages, selected support pathways, monitored progress, and adapted intervention packages,
an axiomatic safety alignment gateway configured to evaluate a generated intervention package against one or more predefined safety, consistency, or non-harm rules prior to delivery,
a multi-model evaluator configured to compare two or more candidate intervention packages according to support, appropriateness, expected completion likelihood, or user suitability,
an adaptive intensity regulator configured to increase, decrease, delay, sequence, suspend, or replace a recommended intervention intensity according to non-completion history, recurrence pattern, or changed user condition, and
an outcome analytics engine configured to generate a pre-intervention state object and a post-intervention state object and to compute one or more change indicators representing reduction, persistence, or escalation of a classified anxiety state,
wherein the system converts anxiety-related user inputs into prioritized action workflows and tracked outcomes.

2. The system of claim 1, wherein the received input data comprise a combination of current user expressions and prior interaction-history inputs retrieved from the user context store.

3. The system of claim 1, wherein the anxiety classification engine is configured to distinguish a transient anxiety state from a recurring anxiety state based on temporal recurrence, prior intervention data, or repeated topic detection.

4. The system of claim 1, wherein the priority engine is configured to generate both a short-term urgency score and a long-term impact score for a classified anxiety category.

5. The system of claim 1, wherein the intervention generation engine is configured to generate a multi-step intervention package comprising at least a reflection action, a concrete action, and a follow-up action, and wherein the intervention generation engine is further configured to produce a plurality of candidate intervention packages from which a preferred intervention package is selected by the multi-model evaluator.

6. The system of claim 1, wherein the routing engine is configured to select among at least a personal-guidance mode, a family-collaboration mode, a community-support mode, a mentor-support mode, and an escalation mode according to the assigned urgency value, actionability value, or relationship-sensitive conditions, and further according to at least one of a prior non-completion record, a recurrence pattern, a trust-related condition, or an escalation-threshold state.

7. The system of claim 1, wherein the follow-up engine is configured to generate a recurring progress prompt in response to incomplete action completion or non-response by the user, wherein the recurring progress prompt is generated by a scheduler that assigns a same-day reminder, a deferred reminder, or a re-engagement trigger condition according to non-completion, delay duration, or repeated unresolved anxiety signals.

8. The system of claim 1, wherein the audit engine is configured to store, in association with the auditable record, an explanation object identifying at least one classification result, priority score, or routing decision that contributed to a generated intervention package, and wherein the auditable record further stores at least one classification result object, priority assignment object, selected support-pathway object, pre-intervention state object, post-intervention state object, and change indicator.

9. A computer-implemented method for converting user anxiety into an action workflow, comprising:
receiving, by one or more processors, input associated with a user concern;
retrieving, from a user context store, structured user records associated with the user;
classifying, by an anxiety classification engine, the input into one or more anxiety categories;
assigning, by a priority engine, at least an urgency value and an actionability value to the one or more anxiety categories;
generating, by an intervention generation engine, an intervention package including one or more actions responsive to the one or more anxiety categories;
evaluating, by an axiomatic safety alignment gateway, whether the intervention package satisfies one or more predefined safety, consistency, or non-harm constraints;
generating, by a multi-model evaluator, a ranking among two or more candidate intervention packages and selecting a preferred intervention package according to said ranking;
selecting, by a routing engine, at least one support pathway for delivery of the intervention package;
tracking, by a follow-up engine, user completion of the one or more actions;
adapting, by an adaptation engine, a subsequent intervention package according to tracked completion and changed user conditions; and
storing, by an audit engine, an auditable record associated with the classifying, assigning, generating, selecting, tracking, and adapting,
wherein adapting the subsequent intervention package further comprises adjusting intervention intensity, sequencing, timing, or substitution according to monitored completion and one or more computed change indicators derived from pre-intervention and post-intervention states.

10. The method of claim 9, wherein the input is associated with schooling, job insecurity, romantic conflict, parenting stress, caregiving burden, financial stress, aging anxiety, or concern relating to death, continuity, or legacy.

11. The method of claim 9, further comprising detecting an escalation threshold based on repeated unresolved anxiety signals or repeated incomplete action completion, wherein the escalation threshold is computed from at least one of a recurrence count, a non-completion count, a delay duration, a category severity score, or a relationship-risk indicator.

12. The method of claim 9, wherein generating the intervention package comprises generating at least a same-day action, a deferred action, and a long-term action, wherein at least one of the same-day action, the deferred action, and the long-term action is selectively suppressed, rescheduled, or replaced by the adaptive intensity regulator in response to detected non-completion or a changed user condition.

13. The method of claim 9, further comprising matching the user with a family member, caregiver, mentor, peer, community channel, or companion-support channel according to the classified anxiety category, and generating one or more collaborative tasks for at least one matched participant according to the classified anxiety category and the selected support pathway.

14. The method of claim 9, further comprising storing intervention-response data in a user-specific memory layer of the user context store, wherein the intervention-response data further comprise a classification result object, a priority assignment object, a selected routing-pathway object, an explanation object, and an auditable record linked to the intervention package.

15. A non-transitory computer-readable medium storing instructions that, when executed by one or more processors, cause the one or more processors to:
receive input associated with user anxiety or uncertainty;
classify the input into one or more anxiety categories;
assign urgency and actionability scores;
generate an action workflow;
route the user to at least one support pathway;
monitor completion of at least one recommended action; and
adapt future recommendations based on monitored completion and updated user state,
wherein the instructions further cause the one or more processors to apply an axiomatic safety alignment gateway, rank a plurality of candidate intervention packages using a multi-model evaluator, regulate intervention intensity using an adaptive intensity regulator, and compute at least one pre-intervention to post-intervention change indicator prior to delivery of a selected intervention package.

16. The non-transitory computer-readable medium of claim 15, wherein the instructions further cause the one or more processors to generate different action workflows for different age groups or lifecycle states.

17. The non-transitory computer-readable medium of claim 15, wherein the instructions further cause the one or more processors to generate an intervention dashboard showing unresolved anxiety categories over time, a current intervention-intensity state, one or more pre-intervention states, one or more post-intervention states, and one or more computed change indicators.

18. The non-transitory computer-readable medium of claim 15, wherein the instructions further cause the one or more processors to generate one or more collaborative tasks for family members associated with the user, wherein the collaborative tasks are separately identified as machine-generated, human-authored, or jointly developed tasks.

19. The non-transitory computer-readable medium of claim 15, wherein the instructions further cause the one or more processors to distinguish companion-generated recommendations, human-authored actions, and jointly developed action plans, and to associate at least one explanation object with each such recommendation, action, or plan.

20. The non-transitory computer-readable medium of claim 15, wherein the instructions further cause the one or more processors to selectively trigger a human review or professional-support escalation pathway, wherein said selective triggering is based on at least one computed escalation threshold derived from recurrence count, non-completion history, or post-intervention state deterioration.
